(19) United States
(12) Patent Application Publication
(10) Pub. No.: US 2017/0252103 A1
(43) Pub. Date: Sep. 7, 2017

(54) AN IRRIGATED ABLATION CATHETER AND PROCESS THEREOF

(71) Applicant: CathRx Ltd, Homebush Bay, NSW (AU)

(72) Inventors: Roman Greifeneder, Bexley (AU); David Ogle, Cowan (AU)

(21) Appl. No.: 15/507,990

(22) PCT Filed: Aug. 28, 2015
(86) PCT No.: PCT/AU2015/000525

§ 371 Date: Mar. 1, 2017

(30) Foreign Application Priority Data
Sep. 15, 2014 (AU) 2014903667

(51) Int. Cl. A61B 18/14

(52) U.S. Cl. CPC A61B 18/1492; A61B 2018/00404

(57) ABSTRACT

An ablation catheter, wherein the catheter includes: a flexible elongated member having a proximal end and a distal end, wherein the elongated member defines an irrigation lumen along its length and the elongated member encapsulates at least one wire; and wherein at least one electrode is attached to the outer surface of the elongated member near to the distal end and the at least one electrode is electrically connected to at least one wire and wherein the at least one electrode includes a plurality of holes that is in fluid communication with the irrigation lumen.

### CROSS-REFERENCE TO RELATED APPLICATIONS

This application is a national phase entry under 35 U.S.C. §371 of International Patent Application PCT/AU2015/000525, filed Aug. 28, 2015, designating the United States of America and published in English as International Patent Publication WO 2016/040982 A1 on Mar. 24, 2016, which claims the benefit under Article 8 of the Patent Cooperation Treaty to Australian Patent Application Serial No. 2014903667, filed Sep. 15, 2014.

### TECHNICAL FIELD

The present invention relates to an irrigated ablation catheter and process for manufacturing the ablation catheter.

### BACKGROUND

Previously, there have been many attempts to develop and market ablation catheters or improved versions of these devices. Typically, ablation catheters of the type described in this specification are suitable for cardiac ablation suitable for the treatment of arrhythmias that medicines or pharmaceuticals typically cannot control or have a limited effect in controlling. Typically, the patient may present with faulty electrical activity in the heart that increases their risk of ventricular fibrillation and sudden cardiac arrest. Catheter-based ablation techniques generally involve advancing flexible catheters into a patient's blood vessels, usually either the femoral vein, internal jugular vein or subclavian vein. The catheters are then advanced toward the heart. Electrical impulses are then used to induce the arrhythmia and local heating or freezing, which is used to ablate (destroy) the abnormal tissue that may be causing the arrhythmia. Catheter ablation is usually performed by an electrophysiologist (a special trained cardiologist) or clinician.

Typically, these types of cardiac ablation catheters are suitable for use in performing procedures including the “Cox maze” procedure wherein surgical ablation is targeted to treat atrial fibrillation wherein the ablation catheter ablates tissues in the atria of the heart.

An example of a previously known ablation catheter is the nMARQ™ device marketed and manufactured by Biosense Webster and described on their website at www.biosensewebster.com/nmarq.php. The nMARQ™ device is described in detail in European Patent No. 2449991. This patent disclosure describes a basic model of an irrigated cardiac ablation catheter.

A further example is disclosed in U.S. Published Patent Application No. 2008/0249522 to Pappone et al., in which is disclosed an irrigated ablation catheter with a flexible tubular body. In this example, solid or rigid electrodes are mounted or positioned along the length of the tubular body.

Any discussion of the prior art throughout the specification should in no way be considered as an admission that such prior art is widely known or forms part of common general knowledge in the field.

### BRIEF SUMMARY

There are several objects of the present disclosure. The present disclosure is directed to improve or alleviate some or all of the problems and issues associated with previously known devices. More specifically, the problems of the previously known devices may include any of the following: rigidity of electrodes, over-ablation at the distal end of the catheter, poor irrigation, rough outer surface of the catheters, or relatively inflexible wiring configurations within the tubular bodies of the catheters.

It is an object of the present disclosure to overcome or ameliorate at least one of the disadvantages of the previously known devices, or to provide a useful alternative.

A first aspect of the present disclosure may relate to an ablation catheter, wherein the catheter includes: a flexible elongated member having a proximal end and a distal end, wherein the elongated member defines an irrigation lumen along its length and the elongated member encapsulates at least one wire and one spacer; and wherein at least one electrode is attached to an outer surface of the elongated member near the distal end and the at least one electrode is electrically connected to the at least one wire, and wherein the at least one electrode includes a plurality of holes, the holes being in fluid communication with the irrigation lumen through the spacer.

Preferably, an electrical current is applied to at least one wire and the catheter ablates tissue proximal to the electrode, when in use. Preferably, an irrigation fluid is pumped into the irrigation lumen and is extruded through the plurality of holes.

The preferred electrode is defined as a ring having a first and second end and a body. Preferably, the first end and second end have rounded edges extending toward a central axis of the body.

An inner surface of the ring and an outer surface of the elongated member may jointly form a cavity. The preferred ring includes holes positioned radially around the outer surface of the ring and, wherein the holes are proximal to the first and second ends. Further, the ring may clamped onto the elongated member and adapted to be secured and engaged on the outer surface of the elongated member. Further, the ring may also be adhered onto the elongated member.

Preferably, the electrode is flexible along a longitudinal axis of the elongated member. The preferred electrode may also comprise an elongated electrical conductive element wrapped helically around the circumference of the elongated member.

The preferred conductive element may include a series of windings and, wherein each neighboring winding includes a gap of no greater than 5 mm. The preferred holes may be formed between the gaps in the windings.

Preferably, the electrode is formed by excising insulative surface portions of the elongated member to expose wire and, wherein the wire forms an electrode.

A first aspect of the present invention may relate to an ablation catheter, wherein the catheter includes:
a flexible elongated member having a proximal end and a distal end, wherein the elongated member defines an irrigation lumen along its length and the elongated member encapsulates a series of wires and a spacer that are helically wound about a longitudinal axis of the irrigation lumen; and
wherein at least one electrode is attached to an outer surface of the elongated member near the distal end and the electrode is electrically connected to at least one wire of the series of wires and, wherein the electrode includes at least one first aperture that is in fluid communication with the irrigation lumen, wherein the first aperture extends from an outer surface of the elongated member through the spacer into the irrigation lumen.

Preferably, the spacer includes a strain relief. The preferred strain relief may be constructed of synthetic fiber.

The preferred outer surface includes at least one second aperture that is adapted to expose a portion of wires. The preferred second aperture is adapted to connect the at least one wire to the respective electrode.

In the context of the present invention, the words “comprise,” “comprising” and the like are to be construed in their inclusive, as opposed to their exclusive, sense, that is in the sense of “including, but not limited to.”

The invention is to be interpreted with reference to at least one of the technical problems described or affiliated with the background art. The present disclosure aims to solve or ameliorate at least one of the technical problems and this may result in one or more advantageous effects as defined by this specification and described in detail with reference to the preferred embodiments of the present invention.

### BRIEF DESCRIPTION OF THE DRAWINGS

FIG. 1 depicts a side perspective view of a first preferred embodiment of a catheter of the present invention;

FIG. 2 depicts a cutaway view of a portion of the first preferred embodiment as shown in FIG. 1 with no electrodes attached;

FIG. 3 depicts a front perspective view of an electrode for use with the first preferred embodiment as shown in FIG. 1;

FIG. 4 depicts a side view of a portion of the first preferred embodiment, wherein the electrode is attached;

FIG. 5 depicts a side view of a second preferred embodiment of the present invention, wherein an alternative electrode has been attached;

FIG. 6 depicts a longitudinal cross-sectional view of the distal end of the catheter forming the first preferred embodiment;

FIG. 7 depicts a longitudinal cross-sectional view of a further preferred embodiment with an alternative electrode configuration;

FIG. 8 depicts a longitudinal cross-sectional view of a further preferred embodiment with an alternative electrode configuration;

FIG. 9 depicts a longitudinal cross-sectional view of the distal end of the catheter forming a further preferred embodiment with another alternative electrode configuration;

FIG. 10 depicts a front perspective view of the distal end of the catheter forming part of the first preferred embodiment, wherein the distal end is in a modified shape;

FIG. 11 depicts a front perspective view of the distal end of the catheter forming part of a further preferred embodiment, wherein the distal end is in a modified shape with alternative electrodes; and

FIG. 12 depicts a front perspective view of the distal end of the catheter forming part of a further preferred embodiment, wherein the distal end is in a modified shape with an alternative electrode.

### DETAILED DESCRIPTION

Preferred embodiments of the invention will now be described with reference to the accompanying drawings and non-limiting examples.

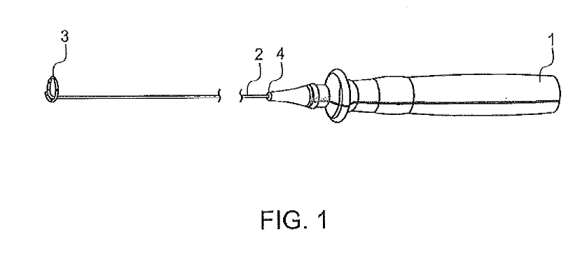

The first preferred embodiment of the present invention is depicted in FIGS. 1-4, 6, and 10. The first preferred embodiment provides for an irrigated ablation catheter comprising: a handle 1, an elongated tubular member 2 having a distal end 3 and a proximal end 4, and, wherein the proximal end 4 may be modified to allow for selective attachment and securing of the handle 1.

Preferably, the tubular member 2 is adapted to be flexible but generally resilient so that the member 2 may be inserted within the blood vessels of the patient and steered by a clinician to its optimal placement for the ablation treatment. The resilient qualities allow the tubular member 2 to be stiff enough to prevent collapse onto itself during insertion or implantation.

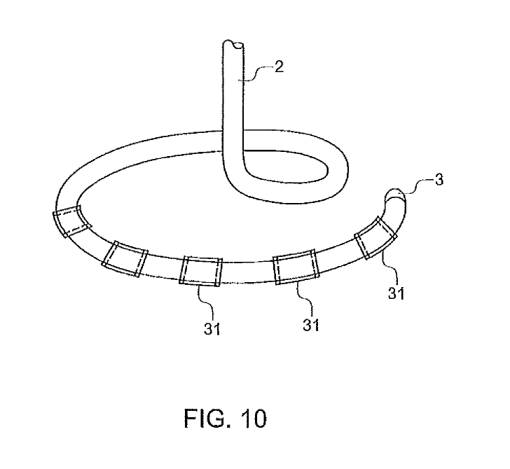

Preferably, the handle 1 may allow for the connection of electrical equipment, power supplies and an irrigation pumping mechanism. The handle 1 may include a series of electrical or fluid connectors at its base (not shown) to facilitate for the electricity and irrigation supplied to the overall system. The handle 1 may be adapted to ergonomically fit a hand of the clinician operating the device. The handle 1 may include features to allow for the steering of the tubular member 2, and also the handle 1 may include a means to allow the tubular member 2 to transition between different shapes at the distal end 3. Preferably, prior to insertion into the patient, the tubular member 2 may be in a linear configuration (not shown) and, wherein the switching means is activated on the handle 1 by the clinician, the distal end 3 may transition into a modified configuration, as shown in FIG. 1 or FIG. 10.

This modified configuration is preferably wherein the distal end 3 is twisted so that the longitudinal axis of the tubular member 2 remains relatively the same and the distal end 3 deviates from the axis at about 90 degrees and then at a predetermined radial length extends around the circumference to the final tip of the distal end 3. This is most clearly visualized with reference to FIG. 10. The modified configuration may be achieved by a use of more rigid stylet inserted along the longitudinal axis of the tubular member 2.

Preferably, the distal end 3 includes at least one electrode mounted, attached or positioned on the outer surface of the tubular member 2. The electrode(s) are adapted to deliver an RF frequency burst to proximal tissue near the electrode when activated by a user or controller mechanism. The RF burst of energy is adapted to destroy or ablate the neighboring tissue in a localized region to allow the clinician to perform Cox maze procedures or similar medical procedures. The catheter of the first preferred embodiment is adapted for use in ablation techniques relating to the ablation of tissue within the atria of the heart, but the device or catheter may be used to ablate other regions or areas as chosen by the respective clinician.

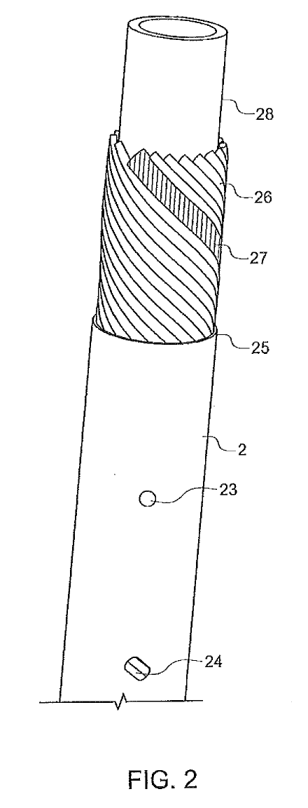

FIG. 2 of the first preferred embodiment depicts a cross-sectional view of the tubular member 2, wherein the electrodes have been removed to allow visual access to the tubular member 2. Preferably, the tubular member 2 includes an irrigation lumen 28 adapted to extend longitudinally through the longitudinal axis of the tubular member 2. The irrigation lumen 28 is adapted to carry and deliver irrigation fluid from the connection in the handle 1 to the distal end 3 and deliver the irrigation fluid to the patient's body at a region proximal to the region of ablation.

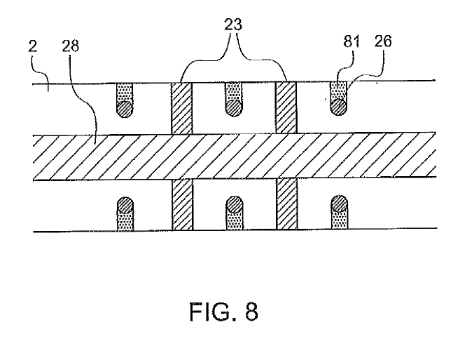

The tubular member 2 may also include a series or a plurality of wires 26. In FIG. 8, there are provided eight sets of two wires which are helically wound around the irrigation lumen 28. Preferably, the helically winding of the wires 26 may allow for the tubular member 2 to be overall more flexible and less likely to accidentally break the wires 26 when in use or when flexed. Incorporated in the winding of the wires 26 is a spacer 27. The spacer 27 may serve several functions and allows for the separation of the series of wires 26 during the helical winding. Preferably, the wires 26 and spacer 27 are encapsulated within an outer flexible sheath 25 to protect the wires.

Preferably, a first aperture 23 or hole may be cut or drilled into the tubular member 2. This first aperture 23 extends into a center of the tubular member 2 through the outer sheath 25 and the spacer 27. The first aperture 23 is adapted to provide fluid communication between the outer surface of the tubular member 2 and the interior of the of the irrigation lumen 28. When irrigation fluid is pumped into the irrigation lumen 28, the irrigation fluid is adapted to flow or exit from the first aperture 23. Preferably, there are multiple first apertures 23 drilled into the outer surface of the tubular member 2 but in FIG. 2, only one is shown for convenience.

A second aperture 24 is preferably cut or drilled into the outer sheath 25 of the tubular member 2. This second aperture 24 is not drilled to the same depth as the first aperture 23 but rather the second aperture 24 exposes one or two of the wires 26 within the tubular member 2 without opening fluid communication with the irrigation lumen 28.

The positioning of the first and second apertures 23, 24 may optimize the positioning of the passages through and into the tubular member 2 without compromising the strength or flexibility of the tubular member 2.

Preferably, the spacer 27 may include or be replaced by a strain relief to assist in limiting over-flex of the tubular member 2, thereby reducing the incidence or likelihood of wire breakage. Preferably, the strain relief may be constructed of Kevlar™ fibers, but other similar materials may be used.

In the preferred tubular member 2, the outer sheath 25 and irrigation lumen 28 may be constructed of silicone-based polymer or PEEK. The preferred construction materials for these items or components should include flexibility and resilience. Also, a preferred material would also be biocompatible for use as an implanted medical device.

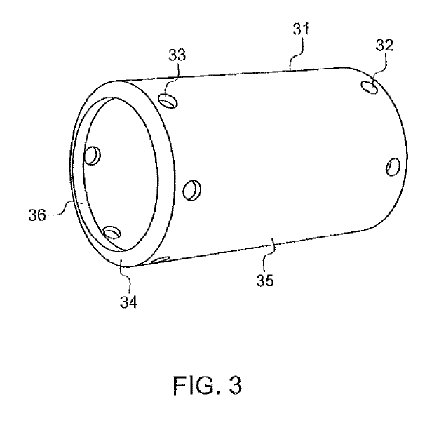

FIG. 3 depicts an electrode 31 adapted to be mounted or positioned on the tubular member 2. The preferred electrode 31 of the first preferred embodiment includes a first and second end joined by a generally cyclindrical body 35. The overall shape of the electrode 31 depicted in FIG. 3 is a generally a ring shape. The first and second end generally includes a rounded or cambered edge. The rounded edge is adapted to extend toward a central axis of the ring electrode. When in use, the rounded edges are adapted to engage or secure the ring electrode 31 against the elongated body of the tubular member 2.

Additionally, the rounded edge extending beyond the inner surface of the body 35 of the ring electrode 31 allows for a cavity to be created between the body 35 and the tubular member 2. The rounded edges may generally prevent the electrodes barbing or catching against portions of the patient's anatomy, when in situ.

Preferably, the ring electrode 31 may include holes 32, 33 positioned radially around an outer surface of the ring electrode 31. The holes 32, 33 are proximal to the first and second ends. In FIG. 3, there are provided six holes at either end of the ring electrode 31, however, other combinations are possible. The diameter and amount of holes in the ring electrode may affect the flow rate and pressure of the irrigation fluid, which is channeled out of the holes, when in use.

Minimizing the number of holes and positioning the holes at either end of the ring electrode may allow for a further reduction of barbing or catching against the anatomy of the patient, when in situ. Further, the minimized number of holes in the ring electrode may generally provide a smoother profile to the exterior surface of the electrode overall.

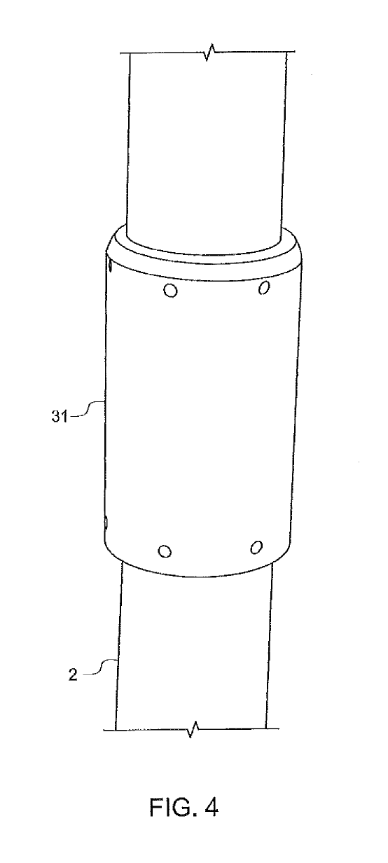

FIG. 4 depicts a ring electrode 31 mounted to the tubular member 2. The ring electrode 31 may be affixed with glue or clamped into positioned by crimping. Preferably, the ends of the ring electrode 31 are adapted to seal against the tubular member 2. Preferably, the second aperture 24 of the tubular member 2 is adapted to engage at least one of the rounded edges or ends of the ring electrode 31 and the first aperture 23 is adapted to be positioned within the cavity, which is formed between the inner surface of the ring electrode 31 and the outer sheath 25 of the tubular member 2. When in use, the irrigation lumen 28 receives irrigation fluid and delivers this fluid to the first aperture 23, which then in turn delivers the fluid into the the cavity. The ring electrode 31 then disperses the fluid across its surface by the series of holes 32, 33 in the body of the ring electrode 31.

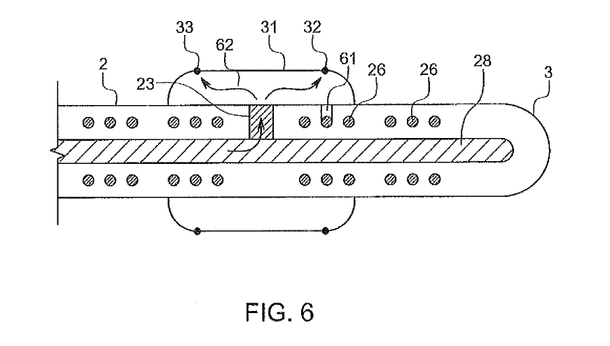

FIG. 6 depicts a cross-sectional view of the fluid flowing in and out of the cavity 62. Further in FIG. 6, the second aperture is shown as region 61. Region 61 has been preferably filled with an electrically conductive polymer or substance. For example, the electrically conductive polymer may be a silver-containing polymer, which is flexible and electrically conductive. The region 61 preferably creates an electrical conduct between the wires 26 and the ring electrode 31. Preferably, the rounded edge or ends of the ring electrode 31 may be crimped, clamped or glued to abut against region 61 to allow for electrical current to be supplied to the ring electrode 31.

In FIG. 10, the tubular member 2 has been distorted into a modified configuration. FIG. 10 allows for visualization of the distal end 3 of the tubular member 2. Prior to implantation, the tubular member 2 may be in a flat or linear configuration, wherein the elongated body of the tubular member 2 is relatively straight and in line. However, when in situ, it may be desirable to alter the shape and configuration of the distal end 3 so that it allows for easier use during the ablation procedures and techniques. The modified configuration shown in FIG. 10 is achieved by the insertion or manipulation of a resilient stylet, which may be adapted to run through the central axis of the tubular member 2. The preferred stylet may use the irrigation lumen 28 (FIG. 2) or have a second separate lumen to be adapted to receive only the stylet. The stylet is preferably deformable to the use moderate finger strength, however, it will return to a predetermined shape once the finger pressure is removed.

The tubular member 2 as shown in FIG. 10 may include multiple ring electrodes 31 positioned proximal to the distal end 3. FIG. 10 depicts five ring electrodes positioned or mounted on the tubular body, but up to ten ring electrodes is generally preferred depending on the wire configurations and the needs of the clinicians. Preferably, the distal end 3 is generally adapted to be deflected from the longitudinal axis of the tubular end of tubular member 2 and then extended out along a radius at a predetermined distance; the distal end 3 is then distorted to encircle and orbit the tubular member 2 at a predetermined distance. The circular portion of the distal end 3 is the preferred location for the ring electrodes 31 so as to provide maximum force against the walls of the area to be ablated.

Preferably, no electrode has been mounted on the most extreme end of the distal end 3 to prevent over-ablation in localized regions.

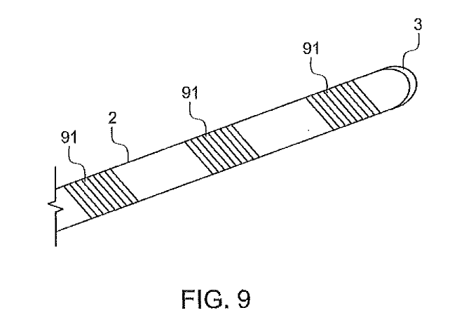

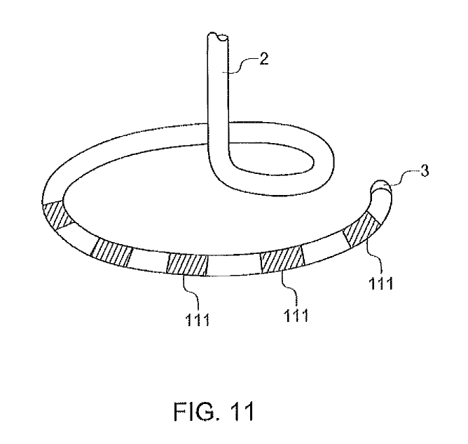

A second preferred embodiment is depicted in FIGS. 9 and 11, wherein the ring electrode 31 has been replaced with a wrapped wire electrode 91. This wrapped wire electrode 91 comprises a length of thicker gauge wire than the wire within the tubular member 2. A length of wire is wrapped tightly in a helical pattern around the circumference of the outer sheath 25 (FIG. 2). Within each electrode 91, the length of wire touches or abuts against a neighboring section of wire from the same length. This length of wrapped wire replaces the ring electrode 31 in respect of ablating features and properties.

Preferably, the wire-wrapped electrode 91 covers and contacts the second aperture, as previously described, and region 61, whereby the electrode 91 is in electrical communication with the wires 26 encapsulated within the tubular member 2.

Additionally, the first aperture 23 is positioned against the under surface of the electrode 91 and delivers irrigation fluid to the general area of the electrode 91. The electrode 91 is generally configured so as to allow or facilitate the exit of this irrigation fluid through small gaps between the wire wrapping forming the electrode 91. The fluid preferably exits via these small gaps between the windings of wire to serve a similar function to the series of holes 32, 33 in the ring electrode 31. Preferably, the conductive element includes a series of windings and, wherein each neighboring winding includes a gap of no greater than 5 mm.

FIG. 11 depicts a similar image to FIG. 10, however, the ring electrodes 31 have been replaced by wire-wrapped electrodes 111.

The wire-wrapped electrodes 111 have several advantages over the ring electrodes 31 and these advantages may include: that the wire-wrapped electrode is flexible along its length, which aids in implantation and use, and the wire-wrapped electrode may be of a continuous length rather than a small rigid electrode as the continuous length of wire wrapping may bend around corners and bends.

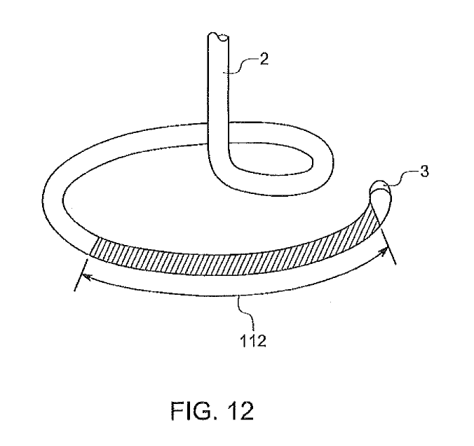

FIG. 12 depicts a wire-wrapped electrode 112 being of indefinite length and, wherein the electrode 112 may extend completely around the circular portion or region of the modified configuration of the distal end 3. This type of configuration may allow for more consistent results and ablations and yet also allows complex catheter geometries to be used.

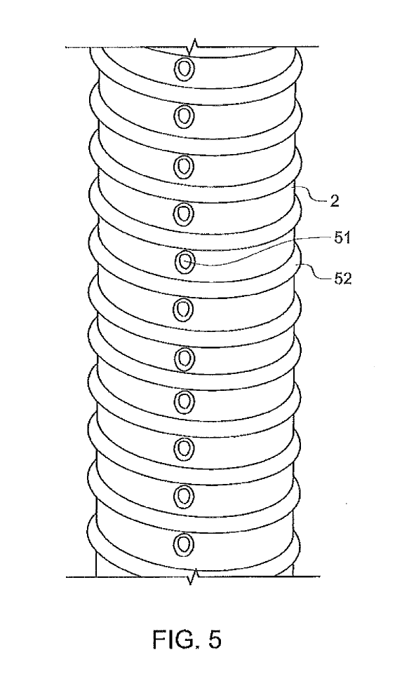

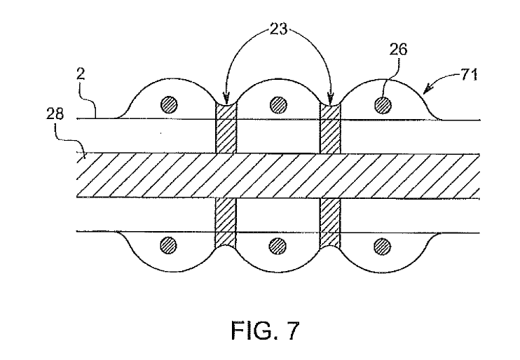

A third preferred embodiment is depicted in reference to FIGS. 5 and 7, wherein wire-wrapped electrode 52 or wires 26 has been used and each coil of the wire wrap has been interleaved with a first aperture to increase to the delivery of irrigation fluid to the localized ablation area. In FIG. 7, the wires 26 have been encapsulated within a relatively thin layer of electrically conductive biocompatible polymer 71 to reduce the impact of the electrode touching the patient. The polymer layer 71 may also allow for the fixing and adhering of the wires 26 to the tubular member 2 to prevent unwanted lateral movement, when in use.

In FIG. 5, the electrode 52 is interleaved with first apertures 51 and the electrode 52 is not encapsulated within a polymer layer 71. This configuration may be easier to manufacture.

In FIG. 8, there is a further alternative design based on the descriptions of FIG. 7, wherein the wires 26 are embedded within the outer sheath 25 (FIG. 2) of the tubular member 2 to provide an overall smoother finish to the body of the catheter. After the wires 26 are embedded within a helical trench 81 around the circumference of the outer sheath 25, the trench 81 is filled with an electrically conductive biocompatible polymer similar to the polymer used in relation to layer 71.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. An ablation catheter, wherein the catheter includes:
a flexible elongated member having a proximal end and a distal end, wherein the elongated member defines an irrigation lumen along its length and the elongated member encapsulates at least one wire and at least one spacer; and
wherein at least one electrode is attached to an outer surface of the elongated member near the distal end and the at least one electrode is electrically connected to at least one wire, and wherein the at least one electrode includes a plurality of holes that are in fluid communication with the irrigation lumen through the at least one spacer.

2. The catheter according to claim 1, wherein an electrical current is applied to at least one wire and the catheter ablates tissue proximal to the at least one electrode, when in use.

3. The catheter according to claim 2, wherein an irrigation fluid is pumped into the irrigation lumen and is extruded through the plurality of holes.

4. The catheter according to claim 3, wherein the at least one electrode is defined as a ring having a first end, a second end and a body.

5. The catheter according to claim 4, wherein the first end and the second end of the ring have rounded edges extending toward a central axis of the body.

6. The catheter according to claim 5, wherein an inner surface of the ring and an outer surface of the elongated member form a cavity.

7. The catheter according to claim 5, wherein the ring includes holes positioned radially around an outer surface of ring, and wherein the holes are proximal to the first end and the second end.

8. The catheter according to claim 4, wherein the ring is clamped onto the elongated member.

9. The catheter according to claim 4, wherein the ring is adhered onto the elongated member.

10. The catheter of claim 1, wherein the at least one electrode is flexible along a longitudinal axis of the elongated member.

11. The catheter of claim 9, wherein the at least one electrode comprises an elongated electrical conductive element wrapped helically around the circumference of the elongated member.

12. The catheter of claim 10, wherein the elongated electrical conductive element includes a series of windings, and wherein each neighboring winding includes a gap of no greater than 5 mm.

13. The catheter of claim 12, wherein the holes are formed between the gaps in the series of windings.

14. The catheter of claim 1, wherein the at least one electrode is formed by excising insulative surface portions of the elongated member to expose the at least one wire and, wherein the at least one wire forms an electrode.

15. An ablation catheter, wherein the catheter includes:
a flexible elongated member having a proximal end and a distal end, wherein the elongated member defines an irrigation lumen along its length and the elongated member encapsulates a series of wires and a spacer that are helically wound about a longitudinal axis of the irrigation lumen; and
wherein at least one electrode is attached to an outer surface of the elongated member near the distal end and the at least one electrode is electrically connected to at least one wire of the series of wires, and wherein the at least one electrode includes at least one first aperture that is in fluid communication with the irrigation lumen, wherein the at least one first aperture extends from an outer surface of the elongated member through the spacer into the irrigation lumen.

16. The catheter of claim 15, wherein the spacer includes a strain relief.

17. The catheter of claim 16, wherein the strain relief is constructed of synthetic fiber.

18. The catheter of claim 15, wherein the outer surface includes at least one second aperture that is adapted to expose a portion of wires.

19. The catheter of claim 18, wherein the at least one second aperture is adapted to connect the at least one wire to the respective electrode.
